(19) United States
(12) Patent Application Publication
(10) Pub. No.: US 2021/0169992 A1
(43) Pub. Date: Jun. 10, 2021

(54) COMPOSITIONS AND METHODS FOR INHIBITING THE PRODUCTION OR ACTIVITY OF D-2HYDROXYGLUTARATE IN SUBJECTS AFFLICTED WITH CANCER

(71) Applicant: President and Fellows of Harvard College, Cambridge, MA (US)

(72) Inventors: Marcia C. Haigis, Winchester, MA (US); Giulia Notarangelo, Cambridge, MA (US)

(21) Appl. No.: 16/951,161

(22) Filed: Nov. 18, 2020

Related U.S. Application Data
(60) Provisional application No. 62/937,354, filed on Nov. 19, 2019.

(51) Int. Cl. A61K 38/44; A61K 31/194; C12N 15/113; A61K 45/06; A61P 35/00

(52) U.S. Cl. CPC A61K 38/443; A61K 31/194; C12N 15/1137; C12N 2310/531; A61P 35/00; C12N 2310/14; C12N 2310/141; A61K 45/06

(57) ABSTRACT

Provided herein are methods and compositions for treating cancer in a subject by administering an agent that inhibits the activity of or decreases the levels of D-2hydroxyglutarate (D-2HG).

### GOVERNMENT SUPPORT

This invention was made with government support under Grant No. DGE1745303, awarded by the National Science Foundation. The government has certain rights in the invention.

### BACKGROUND

Cancer is one of the leading causes of death in the United States. The current standard for treatment is often toxic chemotherapies that do not specifically target cancer cells or malignant tumor. Thus, there is a need to develop new therapies to treat cancer.

### SUMMARY

Provided herein are methods and compositions for treating cancer. In some aspects, the methods disclosed herein comprise administering an agent that inhibits the activity of or decreases the levels of D-2 hydroxyglutarate (D-2HG). Also provided herein are methods of treating cancer in a subject by administering an agent that inhibits the activity of or decreases the levels of an D-2 hydroxyglutarate (D-2HG), wherein the cancer is resistant to at least one isocitrate dehydrogenase (IDH) inhibitor. The IDH inhibitor may be enasidenib or ivosidenib. Also provided herein are methods of increasing the ability of T cells to target cancer cells in a subject with cancer, comprising administering an agent that inhibits the activity of or decreases the levels of D-2hydroxyglutarate (D-2HG).

In some embodiments, the subject has a blood cancer. The cancer cells may express a mutant form of isocitrate dehydrogenase (IDH). The mutant form of isocitrate dehydrogenase IDH may be a gain of function mutation. The mutant form of isocitrate dehydrogenase IDH may be IDH1 R132 and IDH2 R172. The blood cancer may be acute myeloid leukemia (AML). In some embodiments, the subject has a tumor (e.g., a solid tumor, such as a cholangiocarcinoma, a chondrosarcoma, or a glioma). In some embodiments, the tumor expresses a mutant form of isocitrate dehydrogenase (IDH).

The agent may be a small molecule that inhibits the activity of D-2hydroxyglutarate (D-2HG). The agent may be alpha ketoglutarate. The agent may be an interfering nucleic acid specific for an mRNA product of a mutant IDH1 or IDH2 gene, wherein the mutant IDH1 or IDH2 gene encodes for a mutant IDH1 or IDH2 peptide that converts alpha ketoglutarate to D-2 hydroxyglutarate. The mutation in the mutant IDH1 or IDH2 gene may be IDH1 R132 and IDH2 R172. The interfering nucleic acid may be a siRNA, shRNA, miRNA, or a peptide nucleic acid. In some embodiments, the agent decreases the levels of D-2hydroxyglutarate (D-2HG) in a subject by increasing the activity of D-2hydroxyglutarate dehydrogenase. The agent may be a construct (e.g., mRNA construct) that encodes for a D-2hydroxyglutarate dehydrogenase or fragment thereof.

The method may further comprise administering a second agent. The second agent may be a chemotherapeutic agent. The second agent may be an IDH mutant inhibitor or an immune checkpoint inhibitor. The second agent may be a tumor vaccine.

The method may further comprise administering an agent and second agent in different compositions. The second agent may be administered sequentially.

The subject may be a human. The agent may decrease the level of or inhibit the activity of D-2HG by at least 10%, at least 20%, at least 30%, at least 40%, at least 50%, at least 75%, at least 90%, or at least 99%. The agent may be administered to the subject systemically, intravenously, subcutaneously, intramuscularly, orally, or locally to the tumor in the subject.

Also provided herein are methods of determining whether an agent is an anti-cancer therapeutic agent by determining whether the test agent inhibits the activity of or decreases the level of D-2HG, wherein the test agent is determined to be an anti-cancer therapeutic agent if the test agent inhibits the activity of or decreases the level of D-2HG. The test agent may be a library of test agents. The test agent may be, for example, a peptide or a small molecule.

### BRIEF DESCRIPTION OF THE DRAWINGS

FIG. 1 consists of four panels, Panels A-D, and shows D-2HG treatment causes broad changes in T cell metabolism upon activation. Part A shows a schematic of experimental set up. Panels B and C show volcano plots showing metabolites that are differentially upregulated (red) and downregulated (blue) during a time course of T cell activation, where the T cells were treated with and without D-2HG. Part D shows a heat map summarizing the levels of single metabolites in D-2HG treated and control conditions. Log 2 fold change was normalized to the average levels of control group.

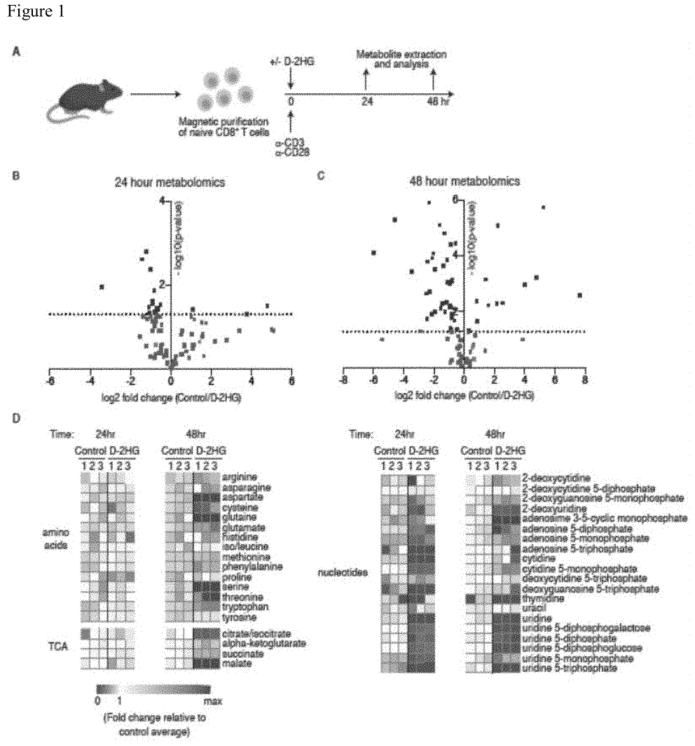

FIG. 2 consists of three panels, Panels A-C, and shows that D-2HG causes accumulation of TCA cycle metabolites. Part A shows the comparison of TCA cycle metabolite levels between treated and control conditions after a 48 hour treatment upon T cell activation. Part B shows that RT-PCR (Panel C) NAD/NADH ratio in control vs. D-2HG treated T cells at 48 hours post-treatment. A 2 day treatment of 1 mM NMN was used as a positive control. *P<0.05, **P<0.01 (Student's t test and two-way ANOVA).

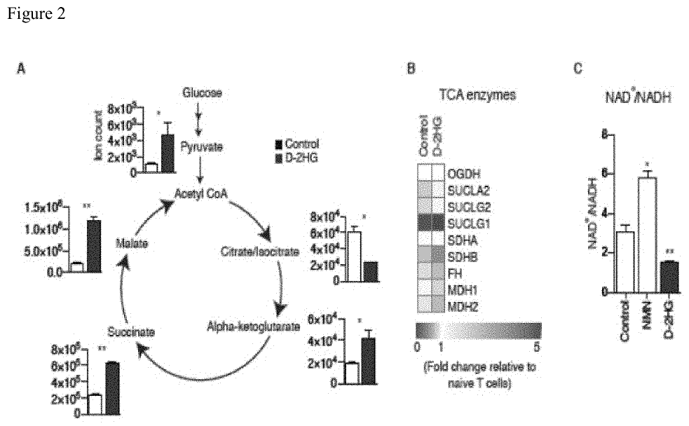

FIG. 3 shows that D-2HG has a mild effect on mitochondrial membrane potential in various cancer cells. Mitochondrial membrane potential was measured by TMRE staining in control, D-2HG, and L-2HG treated GL261 cells.

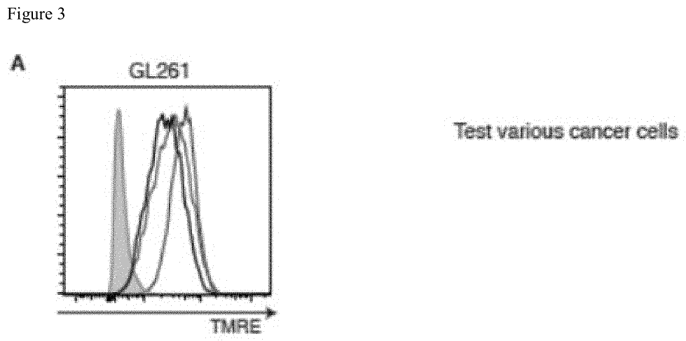

FIG. 4 consists of eight panels, Panels A-H, and shows that mitochondrial membrane potential phenotype is unique to D-2HG. Part A shows the effects of titration of D-2HG on membrane potential, as assessed by TMRE staining. Part B shows that FCCP decouples membrane potential in both control and D-2HG treated conditions. Part C shows that effects of equimolar concentrations of D and L-2HG (20 mM) on membrane potential after a 24 hour treatment. Panel D shows the measurement of basal mitochondrial respiration after acute treatment with D-2HG. Panel E shows the measurement of mitochondrial respiration after acute treatment with D-2HG (1) and subsequent acute treatment with FCCP (2). Panel F shows the measurement of ROS levels following a 24 hour treatment with D-2HG, L-2HG or control. Panel G shows glucose uptake in 48 hour D-2HG or control treated T cells as assessed by 2-NDBG uptake. Panel H shows the measurement of viability following acute glucose starvation in T cells that were activated for 48 hours in the presence or absence of D-2HG. *P<0.05, **P<0.01, ***P<0.001, ****P<0.0001 (Two-way ANOVA).

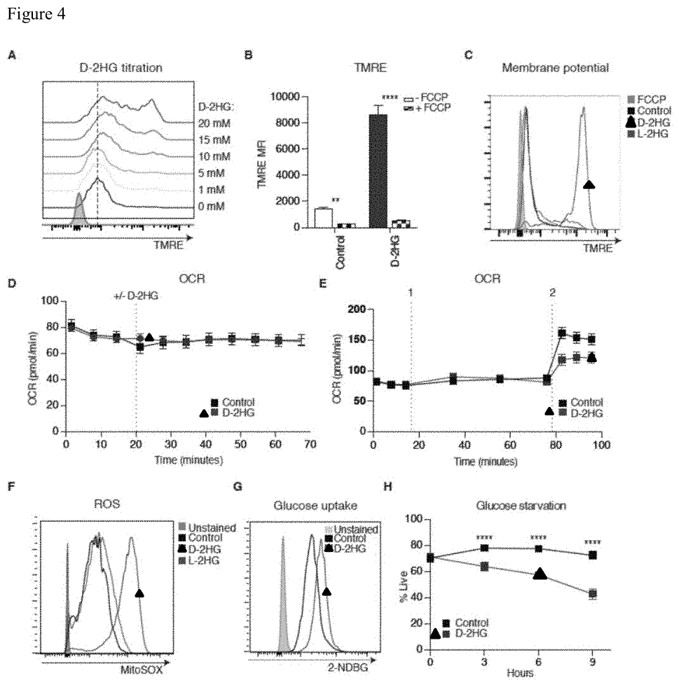

FIG. 5 consists of three panels, Panels A-C, and shows that mitochondrial membrane potential phenotype is not epigenetically driven. HPLC quantification of 5 mC/C levels (Panel A) and 5 hmC/C levels in naïve T cells (Panel B), T cell activated for 24 and 48 hr in the presence or absence of D-2HG, T cells activated for 48 hr in the presence of D-2HG and then D-2HG washed out for 2 hours, and T cells activated for 48 hr in the absence of D-2HG and then D-2HG was acutely introduced for 2 hours. Panel C shows mRNA expression of UCP1, 2 and 3 in T cells activated for 24 hours in the presence or absence of D-2HG.

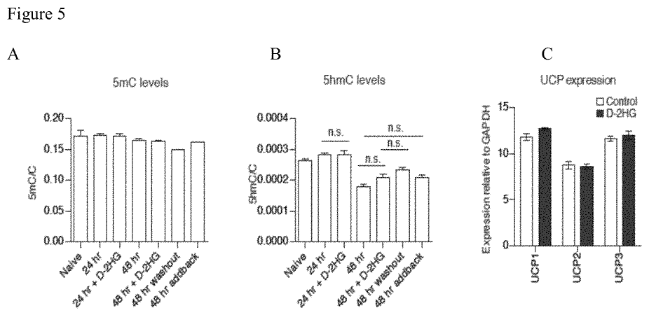

FIG. 6 consists of two panels, Panels A and B, and shows that D-2HG slows down proliferation and promotes apoptosis. Panel A shows the effects of different concentration of D-2HG on T cell proliferation upon activation as assessed by Cell Trace Violet staining. Panel B shows quantitative analysis of apoptosis based on Annexin and PI staining in D-2HG and control cells after 24 hours of treatment. *P<0.05, **P<0.01 (Student's t test).

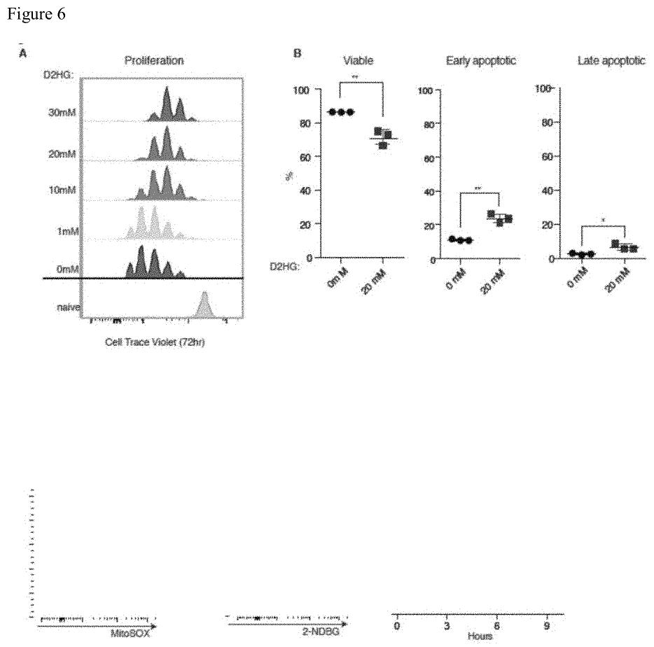

FIG. 7 consists of nine panels, Panels A-I, and shows D-2HG treatment causes mitochondrial hyperpolarization and increased ROS production. Panel A shows time-course of D-2HG uptake in activating T cells. Panel B shows time-course of D-2HG uptake in T cells that have already been activated for 48 hours. Panel C shows membrane potential kinetics as assessed by TMRE staining in activating T cells in the presence or absence of D-2HG. Panel D shows measurement of membrane potential following a 2 hour washout of D-2HG from T cells that had been activated in the presence or absence of the metabolite for 48 hours. Panel E shows measurement of membrane potential following a 2 hour acute addback of D-2HG to T cells that has been activated in the absence of the metabolite for 48 hours. Panel F shows the measurement of oxygen consumption rate in T cells that had been activated for 48 hours and were acutely treated with (1) D-2HG, (2) oligomycin, (3) FCCP, and (4) rotenone and antimycin. Panel G shows a bar graph representation of the oxygen consumption rate dedicated towards maximal respiration of acutely D-2HG treated and control cells. Panel H shows a bar graph representation of the oxygen consumption rate dedicated towards ATP production of acutely D-2HG treated and control cells. Panel I shows the measurement of ROS levels following a 24 hour treatment with D-2HG or control. H₂O₂ and NAC were used as positive and negative controls, respectively. *P<0.05, **P<0.01, ***P<0.001, ****P<0.0001 (Two-way ANOVA).

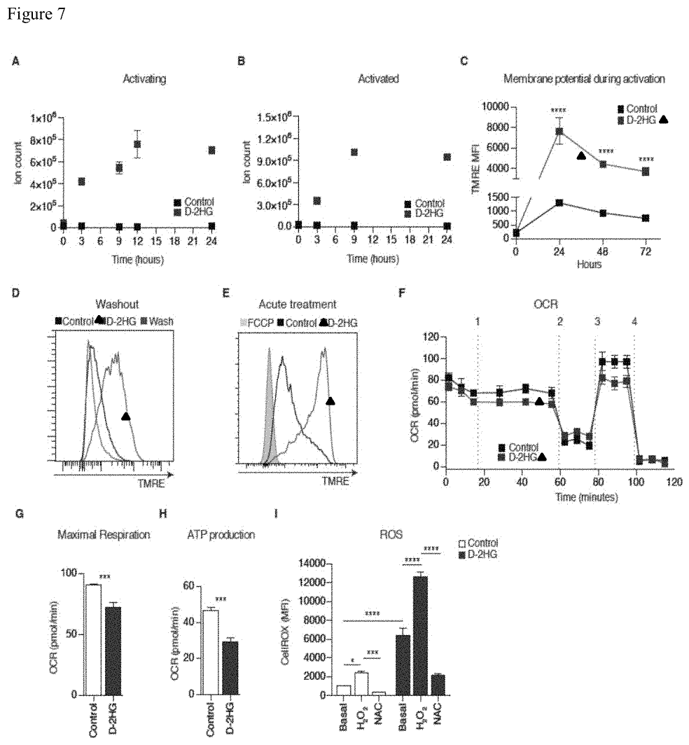

FIG. 8 consists of three panels, Panels A-C, and shows D-2HG is metabolized by D2HGDH and contributes to the mitochondrial membrane potential. Panel A shows isotope tracing of uniformly carbon labeled D-2HG into the TCA cycle. Panel B shows a schematic representation of the targets of the ETC drugs used to study the contribution of each ETC subunit to the mitochondrial membrane potential. Panel C shows the effects of the inhibition of each ETC subunit on the basal membrane potential of D-2HG and control treated T cells. The basal membrane potential of D-2HG and control treated T cells was set to 1.

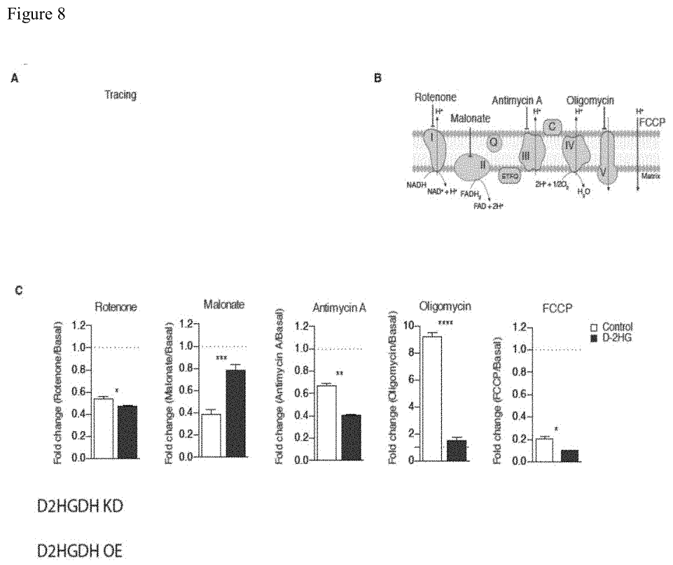

FIG. 9 consists of seven panels, Panels A-G, and shows D-2HG treatment results in the generation of effector T cells with inferior antitumor activity. Panel A shows kinetics of surface expression of markers of activation in the presence or absence of D-2HG. Panel B shows intracellular IL2 staining of T cells activated in the presence of absence of D-2HG. Panel C shows ELISA detection of IL2 secretion from T cells activated in the presence of absence of D-2HG. Data were normalized to cell number. Panel D shows that intracellular IFNγ staining of T cells activated in the presence of absence of D-2HG. Panel E shows ELISA detection of IFNγ secretion from T cells activated in the presence of absence of D-2HG. Data were normalized to cell number. Panel F shows intracellular granzyme staining of T cells activated in the presence of absence of D-2HG. Panel G shows antigen specific killing assay between B16 OVA+ or OVA− tumor cells, IDH mutant B16 OVA+ or OVA− tumor cells, or B16 OVA+ or OVA− supplemented with exogenous D-2HG and OT-1 T cells.

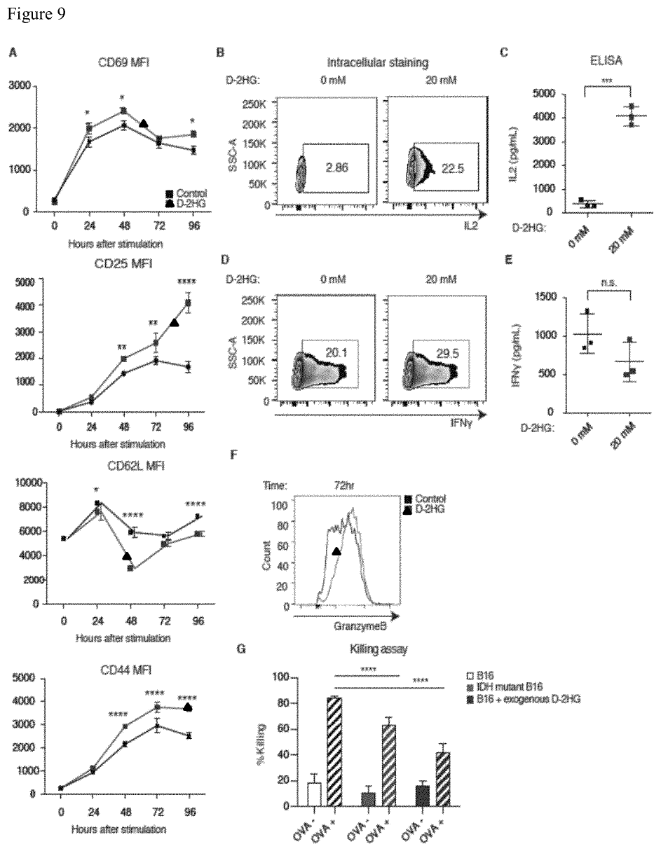

### DETAILED DESCRIPTION

### General

It has been previously thought that D-2HG (D-2hydroxyglutarate) was a byproduct of certain mutant tumors and not taken up/or metabolized directly by cells. It is shown herein that the oncometabolite D-2HG (D-2hydroxyglutarate), which is produced in IDH mutant tumors, is taken up by T cells, alters T cell metabolism, and is itself directly metabolized by T cells.

Provided herein are methods and compositions for treating cancer (e.g., a solid malignant tumor or a blood cancer). Provided herein are methods of treating cancer in a subject by administering a composition (i.e., a composition comprising an agent disclosed herein) that inhibits the activity of or decreases the levels of D-2hydroxyglutarate (D-2HG). Also provided herein are methods of treating cancer in a subject, comprising administering an agent that inhibits the activity of or decreases the levels of an D-2hydroxyglutarate (D-2HG), wherein the cancer is resistant to at least one isocitrate dehydrogenase (IDH) inhibitor (e.g., enasidenib or ivosidenib). In some aspects, provided herein are methods of increasing the ability of T cells to target cancer cells in a subject with cancer by administering an agent that inhibits the activity of or decreases the levels of an D-2hydroxyglutarate (D-2HG). In some embodiments, the agent is alpha ketoglutarate.

### Definitions

For convenience, certain terms employed in the specification, examples, and appended claims are collected here.

As used herein, the term “administering” means providing a pharmaceutical agent or composition to a subject, and includes, but is not limited to, administering by a medical professional and self-administering.

The term “agent” is used herein to denote a chemical compound, a small molecule, a mixture of chemical compounds and/or a biological macromolecule (such as a nucleic acid, an antibody, an antibody fragment, a protein or a peptide). Agents may be identified as having a particular activity by screening assays described herein below. The activity of such agents may render them suitable as a “therapeutic agent” which is a biologically, physiologically, or pharmacologically active substance (or substances) that acts locally or systemically in a subject.

The term “amino acid” is intended to embrace all molecules, whether natural or synthetic, which include both an amino functionality and an acid functionality and capable of being included in a polymer of naturally occurring amino acids. Exemplary amino acids include naturally occurring amino acids; analogs, derivatives and congeners thereof; amino acid analogs having variant side chains; and all stereoisomers of any of the foregoing.

The term “isolated polypeptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The term “isolated nucleic acid” refers to a polynucleotide of genomic, cDNA, or synthetic origin or some combination thereof, which (1) is not associated with the cell in which the “isolated nucleic acid” is found in nature, or (2) is operably linked to a polynucleotide to which it is not linked in nature.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a non-natural arrangement.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein).

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

### Compositions

### Small Molecule Agents

Certain embodiments disclosed herein relate to agents and methods for treating or preventing cancer (e.g., in a subject with a blood cancer or a solid tumor) in a subject comprising administering an agent that inhibits the activity of or decreases the levels of D-2hydroxyglutarate (D-2HG).

The agent may be a small molecule (e.g., alpha ketoglutarate) or pharmaceutically acceptable salts thereof. Additionally, the agents disclosed herein are used in methods of treating cancer (e.g., in a patient with a solid tumor or a blood cancer) in a subject by administering an agent to the subject that inhibits the activity of or decreases the levels of D-2HG. Such agents include those disclosed herein, those known in the art, and those identified using the screening assays described herein.

Agents useful in the methods disclosed herein may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

### Interfering Nucleic Acid Agents

Provided herein are compositions comprising an agent that is an interfering nucleic acid specific for an mRNA product of a mutant IDH1 or IDH2 gene. In some embodiments, the mutant IDH1 or IDH2 gene encodes for a mutant IDH1 or IDH2 peptide that converts alpha ketoglutarate to D-2 hydroxyglutarate (e.g., a gain of function mutation). Examples of such mutations include, but are not limited to, IDH1 R132 and IDH2 R172. The interfering nucleic acid may be a siRNA, shRNA, miRNA, or a peptide nucleic acid. In some embodiments, provided herein are methods of treating cancer by increasing the levels of alpha ketoglutarate by administering to the subject an agent that is an interfering nucleic acid specific for an mRNA product of a mutant IDH1 or IDH2 gene.

Interfering nucleic acids generally include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid: oligomer heteroduplex within the target sequence. Interfering RNA molecules include, but are not limited to, antisense molecules, siRNA molecules, single-stranded siRNA molecules, miRNA molecules and shRNA molecules.

Typically at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acid molecule is double-stranded RNA. The double-stranded RNA molecule may have a 2 nucleotide 3′ overhang. In some embodiments, the two RNA strands are connected via a hairpin structure, forming a shRNA molecule. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues.

Interfering nucleic acid molecules provided herein can contain RNA bases, non-RNA bases or a mixture of RNA bases and non-RNA bases. For example, interfering nucleic acid molecules provided herein can be primarily composed of RNA bases but also contain DNA bases or non-naturally occurring nucleotides.

The interfering nucleic acids can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone. See, e.g., PCT Publication Nos. WO/2013/112053 and WO/2009/008725, incorporated by reference in their entireties.

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm, Buchardt et al. 1993). The backbone of PNAs is formed by peptide bonds rather than phosphodiester bonds, making them well-suited for antisense applications (see structure below). The backbone is uncharged, resulting in PNA/DNA or PNA/RNA duplexes that exhibit greater than normal thermal stability. PNAs are not recognized by nucleases or proteases.

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE™ has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 6,969,766, 7,211,668, 7,022,851, 7,125,994, 7,145,006 and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

Interfering nucleic acids may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-O and the 4′-C positions. LNA enhances backbone preorganization and base stacking to increase hybridization and thermal stability.

The structures of LNAs can be found, for example, in Wengel, et al., Chemical Communications (1998) 455; Tetrahedron (1998) 54:3607, and Accounts of Chem. Research (1999) 32:301); Obika, et al., Tetrahedron Letters (1997) 38:8735; (1998) 39:5401, and Bioorganic Medicinal Chemistry (2008) 16:9230. Compounds provided herein may incorporate one or more LNAs; in some cases, the compounds may be entirely composed of LNAs. Methods for the synthesis of individual LNA nucleoside subunits and their incorporation into oligonucleotides are described, for example, in U.S. Pat. Nos. 7,572,582, 7,569,575, 7,084,125, 7,060,809, 7,053,207, 7,034,133, 6,794,499, and 6,670,461, each of which is incorporated by reference in its entirety. Typical intersubunit linkers include phosphodiester and phosphorothioate moieties; alternatively, non-phosphorous containing linkers may be employed. One embodiment is an LNA containing compound where each LNA subunit is separated by a DNA subunit. Certain compounds are composed of alternating LNA and DNA subunits where the intersubunit linker is phosphorothioate.

“Phosphorothioates” (or S-oligos) are a variant of normal DNA in which one of the nonbridging oxygens is replaced by a sulfur. The sulfurization of the internucleotide bond reduces the action of endo-and exonucleases including 5′ to 3′ and 3′ to 5′ DNA POL 1 exonuclease, nucleases S1 and P1, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-bensodithiol-3-one 1, 1-dioxide (BDTD) (see, e.g., Iyer et al., J. Org. Chem. 55, 4693-4699, 1990). The latter methods avoid the problem of elemental sulfur's insolubility in most organic solvents and the toxicity of carbon disulfide. The TETD and BDTD methods also yield higher purity phosphorothioates.

“2′O-Me oligonucleotides” molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphothioate oligonucleotides (PTOs) for further stabilization. 2′O-Me oligonucleotides (phosphodiester or phosphothioate) can be synthesized according to routine techniques in the art (see, e.g., Yoo et al., Nucleic Acids Res. 32:2008-16, 2004).

The interfering nucleic acids described herein may be contacted with a cell or administered to an organism (e.g., a human). Alternatively, constructs and/or vectors encoding the interfering RNA molecules may be contacted with or introduced into a cell or organism. In certain embodiments, a viral, retroviral or lentiviral vector is used. In some embodiments, the vector has a tropism for cardiac tissue. In some embodiments the vector is an adeno-associated virus.

Typically at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acids contains a 1, 2 or 3 nucleotide mismatch with the target sequence. The interfering nucleic acid molecule may have a 2 nucleotide 3′ overhang. If the interfering nucleic acid molecule is expressed in a cell from a construct, for example from a hairpin molecule or from an inverted repeat of the desired sequence, then the endogenous cellular machinery will create the overhangs. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues.

In some embodiments, the interfering nucleic acid molecule is a siRNA molecule. Such siRNA molecules should include a region of sufficient homology to the target region, and be of sufficient length in terms of nucleotides, such that the siRNA molecule down-regulate target RNA. The term “ribonucleotide” or “nucleotide” can, in the case of a modified RNA or nucleotide surrogate, also refer to a modified nucleotide, or surrogate replacement moiety at one or more positions. It is not necessary that there be perfect complementarity between the siRNA molecule and the target, but the correspondence must be sufficient to enable the siRNA molecule to direct sequence-specific silencing, such as by RNAi cleavage of the target RNA. In some embodiments, the sense strand need only be sufficiently complementary with the antisense strand to maintain the overall double-strand character of the molecule.

In addition, an siRNA molecule may be modified or include nucleoside surrogates. Single stranded regions of an siRNA molecule may be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an siRNA molecule, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

Each strand of an siRNA molecule can be equal to or less than 35, 30, 25, 24, 23, 22, 21, or 20 nucleotides in length. In some embodiments, the strand is at least 19 nucleotides in length. For example, each strand can be between 21 and 25 nucleotides in length. In some embodiments, siRNA agents have a duplex region of 17, 18, 19, 29, 21, 22, 23, 24, or 25 nucleotide pairs, and one or more overhangs, such as one or two 3′ overhangs, of 2-3 nucleotides.

A “small hairpin RNA” or “short hairpin RNA” or “shRNA” includes a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure is cleaved by the cellular machinery into siRNA, which is then bound to the RNA-induced silencing complex (RISC).

In some embodiments, shRNAs are about 15-60, 15-50, or 15-40 (duplex) nucleotides in length, about 15-30, 15-25, or 19-25 (duplex) nucleotides in length, or are about 20-24, 21-22, or 21-23 (duplex) nucleotides in length (e.g., each complementary sequence of the double-stranded shRNA is 15-60, 15-50, 15-40, 15-30, 15-25, or 19-25 nucleotides in length, or about 20-24, 21-22, or 21-23 nucleotides in length, and the double-stranded shRNA is about 15-60, 15-50, 15-40, 15-30, 15-25, or 19-25 base pairs in length, or about 18-22, 19-20, or 19-21 base pairs in length). shRNA duplexes may comprise 3′ overhangs of about 1 to about 4 nucleotides or about 2 to about 3 nucleotides on the antisense strand and/or 5′-phosphate termini on the sense strand. In some embodiments, the shRNA comprises a sense strand and/or antisense strand sequence of from about 15 to about 60 nucleotides in length (e.g., about 15-60, 15-55, 15-50, 15-45, 15-40, 15-35, 15-30, or 15-25 nucleotides in length), or from about 19 to about 40 nucleotides in length (e.g., about 19-40, 19-35, 19-30, or 19-25 nucleotides in length), or from about 19 to about 23 nucleotides in length (e.g., 19, 20, 21, 22, or 23 nucleotides in length).

Non-limiting examples of shRNA include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

Additional embodiments related to the shRNAs, as well as methods of designing and synthesizing such shRNAs, are described in U.S. patent application publication number 2011/0071208, the disclosure of which is herein incorporated by reference in its entirety for all purposes.

In some embodiments, provided herein are micro RNAs (miRNAs). miRNAs represent a large group of small RNAs produced naturally in organisms, some of which regulate the expression of target genes. miRNAs are formed from an approximately 70 nucleotide single-stranded hairpin precursor transcript by Dicer. miRNAs are not translated into proteins, but instead bind to specific messenger RNAs, thereby blocking translation. In some instances, miRNAs base-pair imprecisely with their targets to inhibit translation.

In some embodiments, antisense oligonucleotide compounds are provided herein. In certain embodiments, the degree of complementarity between the target sequence and antisense targeting sequence is sufficient to form a stable duplex. The region of complementarity of the antisense oligonucleotides with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligonucleotide of about 14-15 bases is generally long enough to have a unique complementary sequence.

In certain embodiments, antisense oligonucleotides may be 100% complementary to the target sequence, or may include mismatches, e.g., to improve selective targeting of allele containing the disease-associated mutation, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligonucleotide backbones that are less susceptible to cleavage by nucleases are discussed herein. Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability.

Interfering nucleic acid molecules can be prepared, for example, by chemical synthesis, in vitro transcription, or digestion of long dsRNA by Rnase III or Dicer. These can be introduced into cells by transfection, electroporation, or other methods known in the art. See Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Curr. Opin. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99 (6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99 (9):6047-6052.

In the present methods, an interfering nucleic acid molecule or an interfering nucleic acid encoding polynucleotide can be administered to the subject, for example, as naked nucleic acid, in combination with a delivery reagent, and/or as a nucleic acid comprising sequences that express an interfering nucleic acid molecule. In some embodiments the nucleic acid comprising sequences that express the interfering nucleic acid molecules are delivered within vectors, e.g. plasmid, viral and bacterial vectors. Any nucleic acid delivery method known in the art can be used in the methods described herein. Suitable delivery reagents include, but are not limited to, e.g., the Mirus Transit TKO lipophilic reagent; lipofectin; lipofectamine; cellfectin; polycations (e.g., polylysine), atelocollagen, nanoplexes and liposomes. The use of atelocollagen as a delivery vehicle for nucleic acid molecules is described in Minakuchi et al. Nucleic Acids Res., 32 (13):e109 (2004); Hanai et al. Ann NY Acad Sci., 1082:9-17 (2006); and Kawata et al. Mol Cancer Ther., 7 (9):2904-12 (2008); each of which is incorporated herein in their entirety. Exemplary interfering nucleic acid delivery systems are provided in U.S. Pat. Nos. 8,283,461, 8,313,772, 8,501,930. 8,426,554, 8,268,798 and 8,324,366, each of which is hereby incorporated by reference in its entirety.

In some embodiments of the methods described herein, liposomes are used to deliver an inhibitory oligonucleotide to a subject. Liposomes suitable for use in the methods described herein can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure.

Opsonization-inhibiting moieties for use in preparing the liposomes described herein are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference.

In some embodiments, opsonization inhibiting moieties suitable for modifying liposomes are water-soluble polymers with a number-average molecular weight from about 500 to about 40,000 daltons, or from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. In some embodiments, the opsonization-inhibiting moiety is a PEG, PPG, or derivatives thereof. Liposomes modified with PEG or PEG-derivatives are sometimes called “PEGylated liposomes.”

### Polynucleotide/Nucleic Acid Molecules

Also provided herein are nucleic acid or polynucleotide molecules (RNA constructs) that encode the D-2hydroxyglutarate dehydrogenase, antibodies, antigen binding fragments thereof and/or polypeptides described herein. D-2hydroxyglutarate dehydrogenase converts D-2HG to alpha ketoglutarate. For example, the polynucleotide may encode a D-2 hydroxyglutarate dehydrogenase protein or fragment thereof. The nucleic acids may be present, for example, in whole cells, in a cell lysate, or in a partially purified or substantially pure form. In some embodiments, provided herein are methods of treating cancer by increasing the levels of alpha ketoglutarate by administering to the subject an agent that is a nucleic acid or polynucleotide molecules (RNA constructs) that encode the D-2hydroxyglutarate dehydrogenase.

Nucleic acids described herein can be obtained using standard molecular biology techniques. For example, nucleic acid molecules described herein can be cloned using standard PCR techniques or chemically synthesized. For antibodies obtained from an immunoglobulin gene library (e.g., using phage or yeast display techniques), nucleic acid encoding the antibody can be recovered from the library.

In certain embodiments, provided herein are vectors that contain the isolated nucleic acid molecules described herein. As used herein, the term “vector,” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”). In certain embodiments, provided herein are cells that contain a nucleic acid described herein (e.g., a nucleic acid encoding an antibody, antigen binding fragment thereof, antibody-like molecule, or polypeptide described herein). The cell can be, for example, prokaryotic, eukaryotic, mammalian, avian, murine and/or human.

### Pharmaceutical Compositions

In certain embodiments, provided herein is a composition, e.g., a pharmaceutical composition, containing at least one agent described herein together with a pharmaceutically acceptable carrier. In one embodiment, the composition includes a combination of multiple (e.g., two or more) agents described herein. In some embodiments, the composition further comprises administering a second agent that inhibits the activity of or decreases the levels of D-2HG.

In some embodiments, the agents and/or compositions are delivered systemically or locally. For example, the agents and/or compositions (e.g., pharmaceutical compositions) may be administered to a tumor present in the subject. In some embodiments, the agent or pharmaceutical composition is administered with an additional therapeutic agent (i.e., a second agent). In some embodiments, the agent or pharmaceutical composition is administered with a second cancer therapeutic agent. In some embodiments, the pharmaceutical composition further comprises a second agent for treatment of cancer. In some embodiments, the second agent is a tumor vaccine. In some embodiments, the second agent is an IDH inhibitor. In some embodiments, the additional therapeutic agent is a chemotherapeutic agent. Exemplary chemotherapeutic agents include alkylating agents such as thiotepa and cyclophosphamide (Cytoxan™); alkyl sulfonates such as busulfan, improsulfan and piposulfan; aziridines such as benzodopa, carboquone, meturedopa, and uredopa; emylerumines and memylamelamines including alfretamine, triemylenemelamine, triethylenephosphoramide, triethylenethiophosphoramide, and trimemylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (articularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g., calicheamicin, especially calicheamicin gammall and calicheamicin phili); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK™; razoxane; rhizoxin; sizofiran; spirogermanium; tenuazonic acid; triaziquone; 2,2′,2″-tricUorotriemylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g., paclitaxel (Taxol™, Bristol Meyers Squibb Oncology, Princeton, N.J.) and docetaxel (Taxoteret™, Rhone-Poulenc Rorer, Antony, France); chlorambucil; gemcitabine (Gemzar™); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (Navelbine™); novantrone; teniposide; edatrexate; daunomycin; aminopterin; xeoloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston™); inhibitors of the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™) letrozole (Femara™), and anastrozole (Arimidex™); and anti-androgens such as flutamide, nilutamide, bicalutamide, leuprohde, and goserelin; and pharmaceutically acceptable salts, acids or derivatives of any of the above. In some embodiments, the additional therapeutic agent is an immune checkpoint inhibitor. Immune Checkpoint inhibition broadly refers to inhibiting the checkpoints that cancer cells can produce to prevent or downregulate an immune response. Examples of immune checkpoint proteins are CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, butyrophilins, A2aR, and combinations thereof.

As described in detail below, the pharmaceutical compositions and/or agents disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous, intrathecal, intracerebral or epidural injection as, for example, a sterile solution or suspension, or sustained-release formulation. Methods of preparing pharmaceutical formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, dimethyl sulfoxide (DMSO), polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents provided herein, which may be used in a suitable hydrated form, and/or the pharmaceutical compositions disclosed herein, are formulated into pharmaceutically-acceptable dosage forms by conventional methods known to those of skill in the art. In some embodiments, the agent decreases the level of or inhibits the activity of D-2HG by at least 10%, least 15%, at least 25%, at least 30%, at least 35%, at least 40%, at least 45%, at least 50%, at least 55%, at least 60, %,at least 65%, at least 70%, at least 75%, at least 80%, at least 85%, at least 90%, at least 95%, or at least 99%.

### Indications

In some aspects, provided herein are methods of treating a cancer by administering to a subject (e.g., to a tumor present in a subject) a composition comprising an agent described herein.

In some embodiments, the methods described herein may be used to treat any cancerous or pre-cancerous tumor. In some embodiments, the cancer includes a solid tumor. Cancers that may be treated by methods and compositions provided herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the subject has cancer (e.g., a blood cancer, such as AML). In some embodiments, the cancer comprises a solid tumor. In some embodiments, the tumor is an cholangiocarcinoma, a chondrosarcoma, or a glioma.

In some embodiments, the tumor is an adenocarcinoma, an adrenal tumor, an anal tumor, a bile duct tumor, a bladder tumor, a bone tumor, a blood born tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngreal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

### Methods

Provided herein are methods and compositions related to preventing or treating a condition (e.g., a condition or indication disclosed herein) in a subject comprising administering an agent that inhibits the activity of or decreases the levels of D-2HG.

In some embodiments, the agent decreases the level of or inhibits the activity of D-2HG by at least 5%, by at least 10%, by at least 15%, by at least 20%, by at least 25%, by at least 30%, by at least 35%, by at least 40%, at least 45%, by at least 50%, by at least 55%, by at least 60%, by at least 65%, by at least 70%, by at least 75%, or by at least 80%, by at least 85%, by at least 90%, by at least 95%, or by at least 99%.

The agent may be administered to the subject systemically, intravenously, subcutaneously, intramuscularly.

Also provided herein are methods (e.g., in-vitro methods) of determining whether an agent is a therapeutic agent for cancer comprising determining whether the test agent decreases the levels of or inhibits the activity of D-2HG, wherein the test agent is determined to be an anti-cancer therapeutic agent if the test agent inhibits the activity of or decreases the level of D-2HG. The test agent may be a member of a library of test agents. The test agent may be an interfering nucleic acid, a peptide, a small molecule, or an antibody. The agent may decrease the level of or inhibits the activity of D-2HG by at least 10%, at least 20%, at least 30%, at least 40%, at least 50%, at least 75%, or at least 95%.

Typical subjects for treatment include persons afflicted with or suspected of having or being pre-disposed to a disease disclosed herein, or persons susceptible to, suffering from or that have suffered a disease disclosed herein. A subject may or may not have a genetic predisposition for a disease disclosed herein. In some embodiments, disclosed herein are methods which comprise administration of an agent disclosed herein (e.g., a small molecule or inhibitory nucleic acid) conjointly with a compound for treating a condition disclosed herein. As used herein, the phrase “conjoint administration” refers to any form of administration of two or more different agents (e.g., a composition disclosed herein and a nutrient disclosed herein) such that the second agent is administered while the previously administered agent is still effective in the body. For example, the compositions and/or agents disclosed herein and the nutrients disclosed herein can be administered either in the same formulation or in a separate formulation, either concomitantly or sequentially.

The pharmaceutical compositions disclosed herein may be delivered by any suitable route of administration, including orally, locally, and parenterally. In certain embodiments the pharmaceutical compositions are delivered generally (e.g., via oral or parenteral administration).

In certain aspects, agents and/or compositions disclosed herein may be administered at a dose sufficient to achieve the desired result.

In certain embodiments, the method may comprise administering about 1 ng to about 1 gram of agent or composition to the subject, such as about 1 ng to about 1 μg, about 1 μg to about 1 mg, about 2 μg to about 2 mg, about 3 μg to about 3 mg, about 4 μg to about 4 mg, about 100 μg to about 2 mg, about 200 μg to about 2 mg, about 300 μg to about 3 mg, about 400 μg to about 4 mg, about 250 μg to about 1 mg, or about 250 μg to about 750 μg of the agent or composition. In some embodiments, the method may comprise administering about 25 μg, about 50 μg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 600 μg/kg, about 650 μg/kg, about 700 μg/kg, about 750 μg/kg, about 800 μg/kg, about 850 μg/kg, about 900 μg/kg, about 950 μg/kg, about 1000 μg/kg, about 1200 μg/kg, about 1250 μg/kg, about 1300 μg/kg, about 1333 μg/kg, about 1350 μg/kg, about 1400 μg/kg, about 1500 μg/kg, about 1600 μg/kg, about 1750 μg/kg, about 1800 μg/kg, about 2000 μg/kg, about 2200 μg/kg, about 2250 μg/kg, about 2300 μg/kg, about 2333 μg/kg, about 2350 μg/kg, about 2400 μg/kg, about 2500 μg/kg, about 2667 μg/kg, about 2750 μg/kg, about 2800 μg/kg, about 3 mg/kg, about 3.5 mg/kg, about 3.5 mg/kg, about 4 mg/kg, about 4.5 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, or about 100 mg/kg. In some embodiments, the method may comprise administering about 1 mg/kg to about 10 mg/kg, about 10 mg/kg to about 20 mg/kg, about 20 mg/kg to about 50 mg/kg, about 50 mg/kg to about 100 mg/kg of the agent or composition. The dose may be titrated up down following initial administration to any effective dose.

In some embodiments, administering an agent or composition to the subject comprises administering a bolus of the composition. The method may comprise administering the composition to the subject at least once per month, twice per month, three times per month. In certain embodiments, the method may comprise administering the composition at least once per week, at least once every two weeks, or once every three weeks. In some embodiments, the method may comprise administering the composition to the subject 1, 2, 3, 4, 5, 6, or 7 times per week.

In some embodiments, the agents and/or compositions described herein may be administered conjointly with a second agent (e.g., a second agent disclosed herein).

In certain embodiments, the compositions of the invention can be administered in a variety of conventional ways. In some aspects, the compositions of the invention are suitable for parenteral administration. In some embodiments, these compositions may be administered, for example, intraperitoneally, intravenously, intrarenally, or intrathecally. In some aspects, the compositions of the invention are injected intravenously.

In some embodiments, actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

In general, a suitable daily dose of an agent described herein will be that amount of the agent which is the lowest dose effective to produce a therapeutic effect. Such an effective dose will generally depend upon the factors described above.

Agents useful in the methods disclosed herein may be identified, for example, using assays for screening candidate or test compounds which inhibit the activity of or decreases the level of D-2HG, wherein the test agent is determined to be an anti-cancer therapeutic agent if the test agent inhibits the activity of or decreases the level of D-2HG. The basic principle of the assay systems used to identify compounds that inhibit D-2HG include administering a test compound (e.g., a small molecule) to a system or assay where the activity of a D-2HG and/or the level of a D-2HG may be calculated prior and post administration of a D-2HG inhibitor to the system or assay. In order to test an agent for D-2HG modulatory activity, the reaction mixture is prepared in the presence and absence of the test compound. Control reaction mixtures are incubated without the test compound or with a placebo. The inhibition of D-2HG activity or D-2HG levels is then detected. In some embodiments, the test agent is determined to be a therapeutic agent if the test agent decreases the levels of or inhibits the activity of D-2HG. The test agent may be a member of a library of test agents. The agent may be an interfering nucleic acid, a peptide, a small molecule, an antibody, or any agent disclosed herein. The agent may decreases the level of or inhibits the activity of D-2HG by at least 1%, at least 5%, at least 10%, at least 20%, at least 25%, at least 30%, at least 35%, at least 40%, at least 45%, at least 50%, at least 55%, at least 60%, at least 65%, at least 70% at least 75%, at least 80%, at least 85%, at least 90%, at least 95%, or at least 99%.

### EXEMPLIFICATION

Applicant discovered that the oncometabolite D-2HG (D-2hydroxyglutarate), which is produced in IDH mutant tumors, is taken up by T cells and alters T cell metabolism. Specifically, it is shown herein that D-2HG affects mitochondrial metabolism and electron transport chain (ETC) function. D-2HG treated T cells have their mitochondrial membrane hyperpolarized and their ETC function is impaired under stress conditions. Mechanistically, it is shown herein that this is the result of D-2HG being metabolized and feeding electrons into the ETC. T cells with a hyperpolarized mitochondria are short-lived effector T cells with impaired anti-tumor killing capacities. The data herein also show increased expression of effector markers and reduced killing by D-2HG treated T cells.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### Equivalents

Those skilled in the art will recognize, or be able to ascertain using no more than routine experimentation, many equivalents to the specific embodiments of the invention described herein. Such equivalents are intended to be encompassed by the following claims.

1. A method of treating cancer in a subject, comprising administering an agent that inhibits the activity of or decreases the levels of D-2hydroxyglutarate (D-2HG).

2. The method of claim 1, wherein the cancer is resistant to at least one isocitrate dehydrogenase (IDH) inhibitor.

3. The method of claim 2, wherein the IDH inhibitor is enasidenib or ivosidenib.

4. A method of increasing the ability of T cells to target cancer cells in a subject with cancer, comprising administering an agent that inhibits the activity of or decreases the levels of a D-2hydroxyglutarate (D-2HG).

5. The method of claim 1, wherein the subject has a blood cancer, and the cancer cells express a mutant form of isocitrate dehydrogenase (IDH), wherein the mutant form of isocitrate dehydrogenase IDH is IDH1 R132 and IDH2 R172.

6-7. (canceled)

8. The method of claim 1, wherein the subject has a tumor, and the tumor expresses a mutant form of isocitrate dehydrogenase (IDH).

9-10. (canceled)

11. The method of claim 8, wherein the tumor is a cholangiocarcinoma, a chondrosarcoma, a glioma, an adenocarcinoma, an adrenal tumor, an anal tumor, a bile duct tumor, a bladder tumor, a bone tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngreal tumor, a liver tumor, a lung tumor, a mesothelioma tumor, a multiple myeloma tumor, a muscle tumor, a nasopharyngeal tumor, a nueroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

12. (canceled)

13. The method of claim 1, wherein the agent is alpha keto-glutarate.

14. The method of claim 1, wherein the agent is an interfering nucleic acid specific for an mRNA product of a mutant IDH1 or IDH2 gene, wherein the mutant IDH1 or IDH2 gene encodes for a mutant IDH1 or IDH2 peptide that converts alpha ketoglutarate to D-2 hydroxyglutarate.

15. The method of claim 14, wherein the mutation is IDH1 R132 and IDH2 R172.

16. The method of claim 14, wherein the interfering nucleic acid is a siRNA, shRNA, miRNA, or peptide nucleic acid (PNA).

17-19. (canceled)

20. The method of claim 1, wherein the agent decreases the levels of D-2hydroxyglutarate (D-2HG) in a subject by increasing the activity of D-2hydroxyglutarate dehydrogenase.

21. The method of claim 20, wherein the agent is an mRNA the encodes for a D-2hydroxyglutarate dehydrogenase or fragment thereof.

22. The method of claim 1, wherein the method further comprises administering a second agent comprising a chemotherapeutic agent, an IDH mutant inhibitor, an immune checkpoint inhibitor, or a tumor vaccine.

23-25. (canceled)

26. The method of claim 22, wherein the agent and second agent are administered conjointly.

27. The method of claim 22, wherein the agent and the second agent are administered concomitantly or sequentially.

28. (canceled)

29. The method of claim 1, wherein the agent decreases the level of or inhibits the activity of D-2HG by at least 10%, at least, 20%, at least 30%, at least 40%, at least 50%, at least 75%, at least 90%, or at least 99% .

30-37. (canceled)

38. The method claim 1, wherein the agent is administered intravenously, subcutaneously, intramuscularly, orally, or locally.

39-43. (canceled)

44. A method of determining whether an agent is an anti-cancer therapeutic agent comprising determining whether the test agent inhibits the activity of or decreases the level of D-2HG, wherein the test agent is determined to be an anti-cancer therapeutic agent if the test agent inhibits the activity of or decreases the level of D-2HG.

45. (canceled)

46. The method of claim 44, wherein the test agent is a peptide, small molecule, or an antibody.

47-48. (canceled)

49. The method of claim 44, wherein the test agent inhibits the activity of or decreases the level of listed in D-2HG by at least 50%, at least 60%, at least 70%, at least 80%, at least 90%, or by 100%.

50-54. (canceled)
